(19) United States
(12) Patent Application Publication
(10) Pub. No.: US 2022/0323627 A1
(43) Pub. Date: Oct. 13, 2022

(54) PERSONAL PROTECTION AND PATHOGEN DISINFECTION SYSTEMS AND METHODS

(71) Applicant: SparkCognition, Inc., Austin, TX (US)

(72) Inventor: Syed Mohammad Amir Husain, Georgetown, TX (US)

(21) Appl. No.: 17/659,043

(22) Filed: Apr. 13, 2022

Related U.S. Application Data
(60) Provisional application No. 63/174,340, filed on Apr. 13, 2021.

(51) Int. Cl. A61L 2/24; A61L 2/10

(52) U.S. Cl. CPC A61L 2/24; A61L 2/10

(57) ABSTRACT

A personal protection and pathogen disinfection system includes personal protective equipment (“PPE”) configured to cover at least a portion of a person's face when worn by the person, a disinfection device configured to be worn or carried by the person, an input device configured to receive input from the person, and at least one processor configured to selectively activate the disinfection device responsive to the input.

### CROSS-REFERENCE TO RELATED APPLICATION

The present application claims priority from U.S. Provisional Patent Application No. 63/174,340 filed Apr. 13, 2021, entitled “PERSONAL PROTECTION AND PATHOGEN DISINFECTION SYSTEM,” which is incorporated by reference herein in its entirety.

### FIELD OF THE INVENTION

The present disclosure is generally related to systems and methods for personal protection and pathogen disinfection.

### BACKGROUND

There are many existing and newly discovered infectious diseases that can have serious or fatal repercussions for humans. It is vital to keep safe the personnel most at risk of being exposed to the pathogens that cause such disease. Such personnel include, but are not limited to, employees and contractors at hospitals, clinics, laboratories, and medical research facilities. Protecting those personnel includes accurately and quickly identifying potential infections within a particular environment.

### SUMMARY

The present disclosure describes systems and methods that enable personal protection and pathogen disinfection. In some aspects, a personal protection and pathogen disinfection system includes personal protective equipment (“PPE”) configured to cover at least a portion of a person's face when worn by the person, a disinfection device configured to be worn or carried by the person, an input device configured to receive input from the person, and at least one processor configured to selectively activate the disinfection device responsive to the input.

In some aspects, a method includes receiving input data from an input device, the input data representative of an input from a person at the input device, determining whether to activate a disinfection device configured to be worn or carried by the person based at least on the input data, generating activation data based at least on the determination, and communicating activation data to the disinfection device, the activation data configured to selectively activate the disinfection device.

In some aspects, a computer-readable storage device stores instructions. The instructions, when executed by one or more processors, cause the one or more processors to receive input data from an input device, the input data representative of an input from a person at the input device; determine whether to activate a disinfection device configured to be worn or carried by the person based at least on the input data; generate activation data based at least on the determination; and communicate activation data to the disinfection device, the activation data configured to selectively activate the disinfection device.

In some aspects, a device includes means for receiving input data from an input device, the input data representative of an input from a person at the input device; means for determining whether to activate a disinfection device configured to be worn or carried by the person based at least on the input data; means for generating activation data based at least on the determination; and means for communicating activation data to the disinfection device, the activation data configured to selectively activate the disinfection device.

### BRIEF DESCRIPTION OF THE DRAWINGS

FIG. 1 depicts a system for personal protection and pathogen disinfection in accordance with some examples of the present disclosure.

FIG. 2 depicts a block diagram of a particular implementation of components that may be included in the system of FIG. 1 in accordance with some examples of the present disclosure.

FIG. 3 is a flow chart of an example of a method for personal protection and pathogen disinfection, in accordance with some examples of the present disclosure.

FIG. 4 is an illustrative example of a PPE including a helmet that incorporates aspects of the system of FIG. 1 in accordance with some examples of the present disclosure.

FIG. 5 is an illustrative example of a PPE including a face shield that incorporates aspects of the system of FIG. 1 in accordance with some examples of the present disclosure.

FIG. 6 is an illustrative example of a PPE including a mask or mask cover that incorporates aspects of the system of FIG. 1 in accordance with some examples of the present disclosure.

FIG. 7 is an illustrative example of a headset that incorporates certain aspects of the system of FIG. 1 in accordance with some examples of the present disclosure.

FIG. 8 illustrates an example of a computer system corresponding to the system of FIG. 1 in accordance with some examples of the present disclosure.

### DETAILED DESCRIPTION

Systems and methods are described that enable personal protection and pathogen disinfection. The systems and methods may leverage a combination of machine learning, natural language processing, and one or more augmented reality display(s).

Advantageously, a user of the system is protected from airborne and droplet pathogens while investigating infected persons and surfaces, handling infected material, and disinfecting objects and surfaces using a disinfection device. In a particular aspect, an ultraviolet (“UV”) lamp is part of the system and is controlled based on various user and/or sensor-based input. In other aspects, alternative disinfection mechanisms may be used, as further described herein. In another particular aspect, a computing system is improved through the application of machine learning, natural language processing, and/or augmented reality to the specific computing problem of determining whether to selectively activate a disinfection device, particularly given a likelihood of infection in a particular environment.

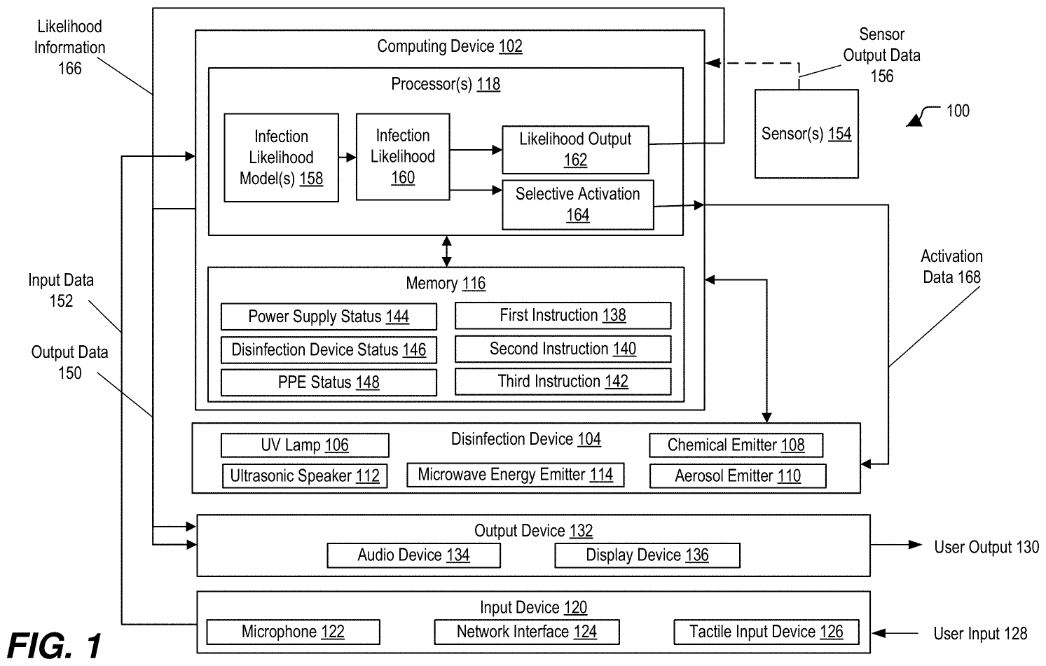

In an illustrative implementation, a system can include one or more personal protective equipment items (“PPE” or “PPEs”) configured to cover at least a portion of a person's face (e.g., a nose and mouth) when the PPE is worn by the person. Examples of the PPE may include, but are not limited to a helmet, a mask or mask cover, a face shield, etc. In some implementations, the system can also include one or more disinfection devices configured to be worn or carried by the person. For example, a disinfection device can include a lamp configured to output ultraviolet (“UV”) light, a chemical emitter, an aerosol emitter, an ultrasonic speaker, a microwave energy emitter, a robotic device, etc., as described in more detail below with reference to FIG. 1. The system can also include one or more input devices configured to receive input from the person. Examples of input device(s) include, but are not limited to, a microphone or microphone array that receives speech input from the user; a button, touchpad, or other input device that receives tactile input from the user; a network interface that receives input via a network from an external device, etc., as described in more detail below with reference to FIG. 1. The system can also include one or more processors configured to perform various functions with respect to the input devices, the disinfection device(s), and the PPE. As an illustrative non-limiting example, the processor(s) may be configured to selectively activate and deactivate a UV lamp based on speech and/or tactile input from the person using the system.

In some implementations, the system can also include one or more sensors. Illustrative examples of such sensors include thermal sensors, infrared sensors, optical sensors or cameras, biosensors, lab-on-chip sensors, airborne particle analysis sensors, etc. The processor(s) in the system may execute various operations based at least in part on sensor data from the sensors. For example, the processor(s) may selectively activate or deactivate the UV lamp based on the sensor data. In a particular aspect, the processor(s) can execute various machine learning models that operate on the sensor data. The models may be used to determine a predicted likelihood that some object or surface within an environment is infected with a pathogen (an “infection likelihood”) and therefore should be disinfected with the disinfection device. When, for example, the infection likelihood exceeds a threshold, the disinfection device can be selectively activated, or the user may be instructed to position the disinfection device in a particular way and then activate the disinfection device. Information based on the infection likelihood may generally be communicated to the user using audio cues (e.g., via speaker) or visual cues (e.g., via an augmented reality heads-up display).

Various methodologies may be used to determine the predicted likelihood of infection. For example, a nano-interferometric biosensor may have bioreceptors tuned to antigens of a particular virus. When a surface or sample (e.g., respiratory fluid sample) is infected, a refractive index of the biosensor is changed (e.g., by a captured virus particle or a chemical reaction due to presence of the virus particle). Light passing through the biosensor as affected by the change in refractive index in a detectable/measurable manner. The measured change in refractive index may be input into a machine learning model to determine, in near-real-time, the predicted likelihood of infection and potentially the specific infectious pathogen(s) in question. As another example, a lateral flow sensor may be coated with antibodies that bind to specific viral proteins, along with a separate coloring agent/antibody. Similar to an at-home pregnancy test, when a specific pathogen is present, the lateral flow sensor may provide colorized visual indicator(s). A computer vision or other machine learning model may determine the infection likelihood based on the size and/or coloring of such indicator(s). In yet another example, lab-on-chip sensor(s) may provide a fast polymerase chain reaction (PCR) with reverse transcription reagent. The lab-on-chip sensor(s) may provide results within thirty minutes, and when the predicted likelihood of infection is high, the user may be instructed to activate the disinfection device and to begin disinfection. As yet another example, a nanotube-based sensor may be used, where a spacing between the nanotubes enables capturing of pathogen (e.g., virus) particles of a known size range. Spectroscopic techniques (e.g., Raman spectroscopy) may be used to identify the pathogen and collect related spectra. In embodiments where spectral techniques are used to collect pathogen-related spectra, the spectra may be input into one or more machine learning classifiers. Examples of such classifiers include, but are not limited to, a support vector machine, a logistic regression model, a decision tree, a random forest algorithm, an artificial neural network, etc. When multiple classifiers are used, ensembling and/or crossvalidation techniques may be applied to determine an overall classification of the pathogen.

Based on the output data from the one or more trained behavior models, activation data and/or likelihood information can be generated, as described in more detail below with reference to FIG. 1. For example, the output data from a trained behavior model may indicate that a surface or object is likely infected with or by a particular pathogen. Information can be sent to an output device to instruct a user to commence disinfection procedure(s), automatically commence disinfection action(s), selectively activate a disinfection device, or take other appropriate corrective action associated with a fix for the infection condition.

In some implementations, multiple infection likelihood models can be generated and scored relative to one another to select an infection detection model to be deployed. Factors used to generate a score for each infection likelihood model and a scoring mechanism used to generate the score can be selected based on data that is to be used to monitor potentially infected objects or surfaces (e.g., the nature or type of sensor data to be used), based on particular goals to be achieved by monitoring (e.g., whether early prediction or a low false positive rate is to be preferred), or based on both.

The described systems and methods address a significant challenge in deploying trained behavior models in pathogen detection environments. As a result, the described systems and methods can provide cost-beneficial monitoring of potentially infected objects and/or surfaces that may not be identical (e.g., operating tables, operating tools, etc.), are located in different environments (e.g., hospitals, schools, battlefields, etc.), are located in hazardous environmental conditions, are exposed to widely different pathogens, etc.

Particular aspects of the present disclosure are described below with reference to the drawings. In the description, common features are designated by common reference numbers throughout the drawings. As used herein, various terminology is used for the purpose of describing particular implementations only and is not intended to be limiting. For example, the singular forms “a,” “an,” and “the” are intended to include the plural forms as well, unless the context clearly indicates otherwise. Further the terms “comprise,” “comprises,” and “comprising” may be used interchangeably with “include,” “includes,” or “including.” Additionally, the term “wherein” may be used interchangeably with “where.” As used herein, “exemplary” may indicate an example, an implementation, and/or an aspect, and should not be construed as limiting or as indicating a preference or a preferred implementation. As used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). As used herein, the term “set” refers to a grouping of one or more elements, and the term “plurality” refers to multiple elements.

In the present disclosure, terms such as “determining,” “calculating,” “estimating,” “shifting,” “adjusting,” etc. may be used to describe how one or more operations are performed. Such terms are not to be construed as limiting and other techniques may be utilized to perform similar operations. Additionally, as referred to herein, “generating,” “calculating,” “estimating,” “using,” “selecting,” “accessing,” and “determining” may be used interchangeably. For example, “generating,” “calculating,” “estimating,” or “determining” a parameter (or a signal) may refer to actively generating, estimating, calculating, or determining the parameter (or the signal) or may refer to using, selecting, or accessing the parameter (or signal) that is already generated, such as by another component or device.

As used herein, “coupled” may include “communicatively coupled,” “electrically coupled,” or “physically coupled,” and may also (or alternatively) include any combinations thereof. Two devices (or components) may be coupled (e.g., communicatively coupled, electrically coupled, or physically coupled) directly or indirectly via one or more other devices, components, wires, buses, networks (e.g., a wired network, a wireless network, or a combination thereof), etc. Two devices (or components) that are electrically coupled may be included in the same device or in different devices and may be connected via electronics, one or more connectors, or inductive coupling, as illustrative, non-limiting examples. In some implementations, two devices (or components) that are communicatively coupled, such as in electrical communication, may send and receive electrical signals (digital signals or analog signals) directly or indirectly, such as via one or more wires, buses, networks, etc. As used herein, “directly coupled” may include two devices that are coupled (e.g., communicatively coupled, electrically coupled, or physically coupled) without intervening components.

As used herein, the term “machine learning” should be understood to have any of its usual and customary meanings within the fields of computers science and data science, such meanings including, for example, processes or techniques by which one or more computers can learn to perform some operation or function without being explicitly programmed to do so. As a typical example, machine learning can be used to enable one or more computers to analyze data to identify patterns in data and generate a result based on the analysis. For certain types of machine learning, the results that are generated include data that indicates an underlying structure or pattern of the data itself. Such techniques, for example, include so called “clustering” techniques, which identify clusters (e.g., groupings of data elements of the data).

For certain types of machine learning, the results that are generated include a data model (also referred to as a “machine-learning model” or simply a “model”). Typically, a model is generated using a first data set to facilitate analysis of a second data set. For example, a first portion of a large body of data may be used to generate a model that can be used to analyze the remaining portion of the large body of data. As another example, a set of historical data can be used to generate a model that can be used to analyze future data.

Since a model can be used to evaluate a set of data that is distinct from the data used to generate the model, the model can be viewed as a type of software (e.g., instructions, parameters, or both) that is automatically generated by the computer(s) during the machine learning process. As such, the model can be portable (e.g., can be generated at a first computer, and subsequently moved to a second computer for further training, for use, or both). Additionally, a model can be used in combination with one or more other models to perform a desired analysis. To illustrate, first data can be provided as input to a first model to generate first model output data, which can be provided (alone, with the first data, or with other data) as input to a second model to generate second model output data indicating a result of a desired analysis. Depending on the analysis and data involved, different combinations of models may be used to generate such results. In some examples, multiple models may provide model output that is input to a single model. In some examples, a single model provides model output to multiple models as input.

Examples of machine-learning models include, without limitation, perceptrons, neural networks, support vector machines, regression models, decision trees, Bayesian models, Boltzmann machines, adaptive neuro-fuzzy inference systems, as well as combinations, ensembles and variants of these and other types of models. Variants of neural networks include, for example and without limitation, prototypical networks, autoencoders, transformers, self-attention networks, convolutional neural networks, deep neural networks, deep belief networks, etc. Variants of decision trees include, for example and without limitation, random forests, boosted decision trees, etc.

Since machine-learning models are generated by computer(s) based on input data, machine-learning models can be discussed in terms of at least two distinct time windows: a creation/training phase and a runtime phase. During the creation/training phase, a model is created, trained, adapted, validated, or otherwise configured by the computer based on the input data (which in the creation/training phase, is generally referred to as “training data”). Note that the trained model corresponds to software that has been generated and/or refined during the creation/training phase to perform particular operations, such as classification, prediction, encoding, or other data analysis or data synthesis operations. During the runtime phase (or “inference” phase), the model is used to analyze input data to generate model output. The content of the model output depends on the type of model. For example, a model can be trained to perform classification tasks or regression tasks, as non-limiting examples. In some implementations, a model may be continuously, periodically, or occasionally updated, in which case training time and runtime may be interleaved or one version of the model can be used for inference while a copy is updated, after which the updated copy may be deployed for inference.

In some implementations, a previously generated model is trained (or re-trained) using a machine-learning technique. In this context, “training” refers to adapting the model or parameters of the model to a particular data set. Unless otherwise clear from the specific context, the term “training” as used herein includes “re-training” or refining a model for a specific data set. For example, training may include so called “transfer learning.” As described further below, in transfer learning a base model may be trained using a generic or typical data set, and the base model may be subsequently refined (e.g., re-trained or further trained) using a more specific data set.

A data set used during training is referred to as a “training data set” or simply “training data”. The data set may be labeled or unlabeled. “Labeled data” refers to data that has been assigned a categorical label indicating a group or category with which the data is associated, and “unlabeled data” refers to data that is not labeled. Typically, “supervised machine-learning processes” use labeled data to train a machine-learning model, and “unsupervised machine-learning processes” use unlabeled data to train a machine-learning model; however, it should be understood that a label associated with data is itself merely another data element that can be used in any appropriate machine-learning process. To illustrate, many clustering operations can operate using unlabeled data; however, such a clustering operation can use labeled data by ignoring labels assigned to data or by treating the labels the same as other data elements.

Machine-learning models can be initialized from scratch (e.g., by a user, such as a data scientist) or using a guided process (e.g., using a template or previously built model). Initializing the model includes specifying parameters and hyperparameters of the model. “Hyperparameters” are characteristics of a model that are not modified during training, and “parameters” of the model are characteristics of the model that are modified during training. The term “hyperparameters” may also be used to refer to parameters of the training process itself, such as a learning rate of the training process. In some examples, the hyperparameters of the model are specified based on the task the model is being created for, such as the type of data the model is to use, the goal of the model (e.g., classification, regression, infection detection), etc. The hyperparameters may also be specified based on other design goals associated with the model, such as a memory footprint limit, where and when the model is to be used, etc.

Model type and model architecture of a model illustrate a distinction between model generation and model training. The model type of a model, the model architecture of the model, or both, can be specified by a user or can be automatically determined by a computing device. However, neither the model type nor the model architecture of a particular model is changed during training of the particular model. Thus, the model type and model architecture are hyperparameters of the model and specifying the model type and model architecture is an aspect of model generation (rather than an aspect of model training). In this context, a “model type” refers to the specific type or sub-type of the machine-learning model. As noted above, examples of machine-learning model types include, without limitation, perceptrons, neural networks, support vector machines, regression models, decision trees, Bayesian models, Boltzmann machines, adaptive neuro-fuzzy inference systems, as well as combinations, ensembles and variants of these and other types of models. In this context, “model architecture” (or simply “architecture”) refers to the number and arrangement of model components, such as nodes or layers, of a model, and which model components provide data to or receive data from other model components. As a non-limiting example, the architecture of a neural network may be specified in terms of nodes and links. To illustrate, a neural network architecture may specify the number of nodes in an input layer of the neural network, the number of hidden layers of the neural network, the number of nodes in each hidden layer, the number of nodes of an output layer, and which nodes are connected to other nodes (e.g., to provide input or receive output). As another non-limiting example, the architecture of a neural network may be specified in terms of layers. To illustrate, the neural network architecture may specify the number and arrangement of specific types of functional layers, such as long-short-term memory (“LSTM”) layers, fully connected (“FC”) layers, convolution layers, etc. While the architecture of a neural network implicitly or explicitly describes links between nodes or layers, the architecture does not specify link weights. Rather, link weights are parameters of a model (rather than hyperparameters of the model) and are modified during training of the model.

In many implementations, a data scientist selects the model type before training begins. However, in some implementations, a user may specify one or more goals (e.g., classification or regression), and automated tools may select one or more model types that are compatible with the specified goal(s). In such implementations, more than one model type may be selected, and one or more models of each selected model type can be generated and trained. A best performing model (based on specified criteria) can be selected from among the models representing the various model types. Note that in this process, no particular model type is specified in advance by the user, yet the models are trained according to their respective model types. Thus, the model type of any particular model does not change during training.

Similarly, in some implementations, the model architecture is specified in advance (e.g., by a data scientist); whereas in other implementations, a process that both generates and trains a model is used. Generating (or generating and training) the model using one or more machine-learning techniques is referred to herein as “automated model building”. In one example of automated model building, an initial set of candidate models is selected or generated, and then one or more of the candidate models are trained and evaluated. In some implementations, after one or more rounds of changing hyperparameters and/or parameters of the candidate model(s), one or more of the candidate models may be selected for deployment (e.g., for use in a runtime phase).

Certain aspects of an automated model building process may be defined in advance (e.g., based on user settings, default values, or heuristic analysis of a training data set) and other aspects of the automated model building process may be determined using a randomized process. For example, the architectures of one or more models of the initial set of models can be determined randomly within predefined limits. As another example, a termination condition may be specified by the user or based on configurations settings. The termination condition indicates when the automated model building process should stop. To illustrate, a termination condition may indicate a maximum number of iterations of the automated model building process, in which case the automated model building process stops when an iteration counter reaches a specified value. As another illustrative example, a termination condition may indicate that the automated model building process should stop when a reliability metric associated with a particular model satisfies a threshold. As yet another illustrative example, a termination condition may indicate that the automated model building process should stop if a metric that indicates improvement of one or more models over time (e.g., between iterations) satisfies a threshold. In some implementations, multiple termination conditions, such as an iteration count condition, a time limit condition, and a rate of improvement condition can be specified, and the automated model building process can stop when one or more of these conditions is satisfied.

Another example of training a previously generated model is transfer learning. “Transfer learning” refers to initializing a model for a particular data set using a model that was trained using a different data set. For example, a “general purpose” model can be trained to detect anomalies in vibration data associated with a variety of types of rotary equipment, and the general-purpose model can be used as the starting point to train a model for one or more specific types of rotary equipment, such as a first model for generators and a second model for pumps. As another example, a general-purpose natural-language processing model can be trained using a large selection of natural-language text in one or more target languages. In this example, the general-purpose natural-language processing model can be used as a starting point to train one or more models for specific natural-language processing tasks, such as translation between two languages, question answering, or classifying the subject matter of documents. Often, transfer learning can converge to a useful model more quickly than building and training the model from scratch.

Training a model based on a training data set generally involves changing parameters of the model with a goal of causing the output of the model to have particular characteristics based on data input to the model. To distinguish from model generation operations, model training may be referred to herein as optimization or optimization training. In this context, “optimization” refers to improving a metric, and does not mean finding an ideal (e.g., global maximum or global minimum) value of the metric. Examples of optimization trainers include, without limitation, backpropagation trainers, derivative free optimizers (DFOs), and extreme learning machines (ELMs). As one example of training a model, during supervised training of a neural network, an input data sample is associated with a label. When the input data sample is provided to the model, the model generates output data, which is compared to the label associated with the input data sample to generate an error value. Parameters of the model are modified in an attempt to reduce (e.g., optimize) the error value. As another example of training a model, during unsupervised training of an autoencoder, a data sample is provided as input to the autoencoder, and the autoencoder reduces the dimensionality of the data sample (which is a lossy operation) and attempts to reconstruct the data sample as output data. In this example, the output data is compared to the input data sample to generate a reconstruction loss, and parameters of the autoencoder are modified in an attempt to reduce (e.g., optimize) the reconstruction loss.

As another example, to use supervised training to train a model to perform a classification task, each data element of a training data set may be labeled to indicate a category or categories to which the data element belongs. In this example, during the creation/training phase, data elements are input to the model being trained, and the model generates output indicating categories to which the model assigns the data elements. The category labels associated with the data elements are compared to the categories assigned by the model. The computer modifies the model until the model accurately and reliably (e.g., within some specified criteria) assigns the correct labels to the data elements. In this example, the model can subsequently be used (in a runtime phase) to receive unknown (e.g., unlabeled) data elements, and assign labels to the unknown data elements. In an unsupervised training scenario, the labels may be omitted. During the creation/training phase, model parameters may be tuned by the training algorithm in use such that during the runtime phase, the model is configured to determine which of multiple unlabeled “clusters” an input data sample is most likely to belong to.

As another example, to train a model to perform a regression task, during the creation/training phase, one or more data elements of the training data are input to the model being trained, and the model generates output indicating a predicted value of one or more other data elements of the training data. The predicted values of the training data are compared to corresponding actual values of the training data, and the computer modifies the model until the model accurately and reliably (e.g., within some specified criteria) predicts values of the training data. In this example, the model can subsequently be used (in a runtime phase) to receive data elements and predict values that have not been received. To illustrate, the model can analyze time series data, in which case, the model can predict one or more future values of the time series based on one or more prior values of the time series.

In some aspects, the output of a model can be subjected to further analysis operations to generate a desired result. To illustrate, in response to particular input data, a classification model (e.g., a model trained to perform classification tasks) may generate output including an array of classification scores, such as one score per classification category that the model is trained to assign. Each score is indicative of a likelihood (based on the model's analysis) that the particular input data should be assigned to the respective category. In this illustrative example, the output of the model may be subjected to a softmax operation to convert the output to a probability distribution indicating, for each category label, a probability that the input data should be assigned the corresponding label. In some implementations, the probability distribution may be further processed to generate a one-hot encoded array. In other examples, other operations that retain one or more category labels and a likelihood value associated with each of the one or more category labels can be used.

One example of a machine-learning model is an autoencoder. An autoencoder is a particular type of neural network that is trained to receive multivariate input data, to process at least a subset of the multivariate input data via one or more hidden layers, and to perform operations to reconstruct the multivariate input data using output of the hidden layers. If at least one hidden layer of an autoencoder includes fewer nodes than the input layer of the autoencoder, the autoencoder may be referred to herein as a dimensional reduction model. If each of the one or more hidden layer(s) of the autoencoder includes more nodes than the input layer of the autoencoder, the autoencoder may be referred to herein as a denoising model or a sparse model, as explained further below.

For dimensional reduction type autoencoders, the hidden layer with the fewest nodes is referred to as the latent space layer. Thus, a dimensional reduction autoencoder is trained to receive multivariate input data, to perform operations to dimensionally reduce the multivariate input data to generate latent space data in the latent space layer, and to perform operations to reconstruct the multivariate input data using the latent space data. “Dimensional reduction” in this context refers to representing n values of multivariate input data using z values (e.g., as latent space data), where n and z are integers and z is less than n. Often, in an autoencoder the z values of the latent space data are then dimensionally expanded to generate n values of output data. In some special cases, a dimensional reduction model may generate m values of output data, where m is an integer that is not equal to n. As used herein, such special cases are still referred to as autoencoders as long as the data values represented by the input data are a subset of the data values represented by the output data or the data values represented by the output data are a subset of the data values represented by the input data. For example, if the multivariate input data includes ten sensor data values from ten sensors, and the dimensional reduction model is trained to generate output data representing only five sensor data values corresponding to five of the ten sensors, then the dimensional reduction model is referred to herein as an autoencoder. As another example, if the multivariate input data includes ten sensor data values from ten sensors, and the dimensional reduction model is trained to generate output data representing ten sensor data values corresponding to the ten sensors and to generate a variance value (or other statistical metric) for each of the sensor data values, then the dimensional reduction model is also referred to herein as an autoencoder (e.g., a variational autoencoder).

Denoising autoencoders and sparse autoencoders do not include a latent space layer to force changes in the input data. An autoencoder without a latent space layer could simply pass the input data, unchanged, to the output nodes resulting in a model with little utility. Denoising autoencoders avoid this result by zeroing out a subset of values of an input data set while training the denoising autoencoder to reproduce the entire input data set at the output nodes. Put another way, the denoising autoencoder is trained to reproduce an entire input data sample based on input data that includes less than the entire input data sample. For example, during training of a denoising autoencoder that includes 10 nodes in the input layer and 10 nodes in the output layer, a single set of input data values includes 10 data values; however, only a subset of the 10 data values (e.g., between 2 and 9 data values) are provided to the input layer. The remaining data values are zeroed out. To illustrate, out of ten data values, seven data values may be provided to a respective seven nodes of the input layer, and zero values may be provided to the other three nodes of the input layer. Fitness of the denoising autoencoder is evaluated based on how well the output layer reproduces all ten data values of the set of input data values, and during training, parameters of the denoising autoencoder are modified over multiple iterations to improve its fitness.

Sparse autoencoders prevent passing the input data unchanged to the output nodes by selectively activating a subset of nodes of one or more of the hidden layers of the sparse autoencoder. For example, if a particular hidden layer has ten nodes, only three nodes may be activated for particular data. The sparse autoencoder is trained such that which nodes are activated is data dependent. For example, for a first data sample, three nodes of the particular hidden layer may be activated, whereas for a second data sample, five nodes of the particular hidden layer may be activated.

One use case for autoencoders is detecting significant changes in data. For example, an autoencoder can be trained using training sensor data gathered while a monitored system is operating in a first operational mode. In this example, after the autoencoder is trained, real-time sensor data from the monitored system can be provided as input data to the autoencoder. If the real-time sensor data is sufficiently similar to the training sensor data, then the output of the autoencoder should be similar to the input data. Illustrated mathematically:

[symbol]

−xₖ≈0

where [symbol] represents an output data value k and xₖ represents the input data value k. If the output of the autoencoder exactly reproduces the input, then: [symbol] −xₖ=0 for each data value k. However, it is generally the case that the output of a well-trained autoencoder is not identical to the input. In such cases, [symbol] −xₖ=rₖ, where rₖ represents a residual value. Residual values that result when particular input data is provided to the autoencoder can be used to determine whether the input data is similar to training data used to train the autoencoder. For example, when the input data is similar to the training data, relatively small residual values should result. In contrast, when the input data is not similar to the training data, relatively large residual values should result. During runtime operation, residual values calculated based on output of the autoencoder can be used to determine the likelihood or risk that the input data differs significantly from the training data.

As one particular example, the input data can include multivariate sensor data representing monitored parameters of a potentially infected environment. In this example, the autoencoder can be trained using training data gathered while the environment was being monitored in a first operational mode (e.g., a normal mode or some other mode). During use, real-time sensor data from the monitored system can be input to the autoencoder, and residual values can be determined based on differences between the real-time sensor data and output data from the autoencoder. If the monitored environment transitions to a second operational mode (e.g., an abnormal mode, a second normal mode, or some other mode) statistical properties of the residual values (e.g., the mean or variance of the residual values over time) will change. Detection of such changes in the residual values can provide an early indication of changes associated with the monitored environment. To illustrate, one use of the example above is early detection of potential pathogen infection within the monitored environment. In this use case, the training data includes a variety of data samples representing one or more “normal” operating modes. During runtime, the input data to the autoencoder represents the current (e.g., real-time) sensor data values, and the residual values generated during runtime are used to detect early onset of an abnormal operating mode. In other use cases, autoencoders can be trained to detect changes between two or more different normal operating modes (in addition to, or instead of, detecting onset of abnormal operating modes).

Further, in some implementations a user of the instant system can provide speech input. In such implementations, one or more natural language processing (“NLP”) models can be executed by the processor(s) to analyze the user's speech and determine what the user is saying and how to respond to the user's speech (e.g., “turn on lamp,” “turn off lamp,” “battery status,” “date check,” “time check,” “alert teammate,” “how much longer until disinfection is complete,” etc.).

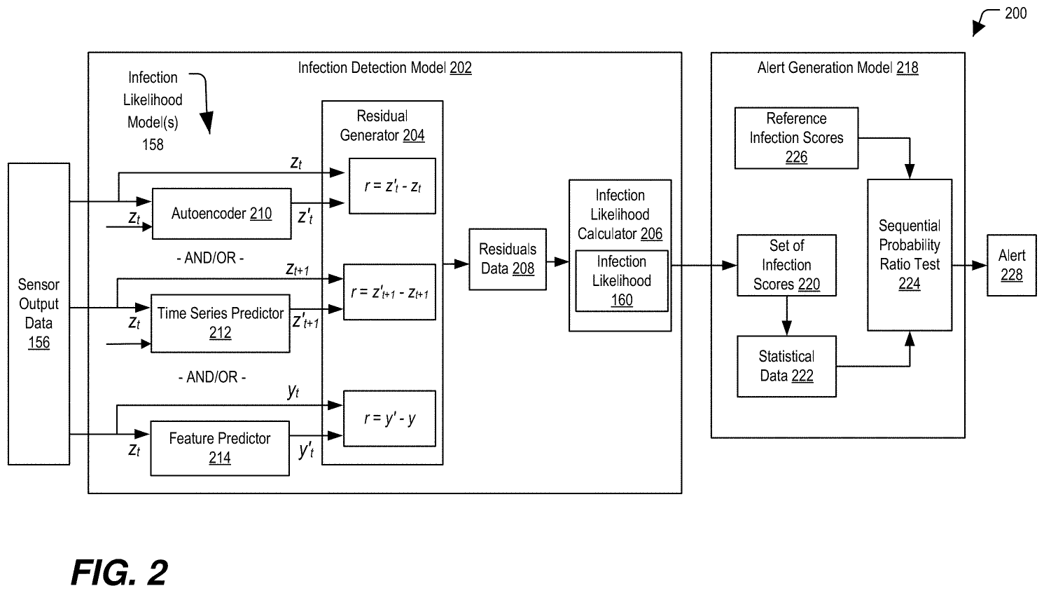

FIG. 1 depicts a system 100 for personal protection and pathogen disinfection in accordance with some examples of the present disclosure. In some implementations, one or more components of the system 100 can be part of one or more items of personal protective equipment (“PPE” or “PPEs”), as described in more detail below and with reference to FIGS. 2-8. For example, the system 100 includes a disinfection device 104 configured to be worn or carried by a person. In some implementations, the person has at least a portion of their face covered by one or more PPEs. The disinfection device 104 can be any device configured to be worn or carried by the person as a separate device, integrated into another device worn or carried by the person (e.g., as a ring, bracelet, lanyard, etc.), external to the PPE, integrated into the PPE, etc. The disinfection device 104 is configured to engage one or more disinfection operations designed to detect, diagnose, disinfect, warn, or otherwise operate to protect a person within a potentially infected environment.

In a particular implementation, the disinfection device 104 can include a lamp 106 configured to output UV light. The UV lamp 106 may be portable and worn or carried by the person. For example, the UV lamp 106 may be handheld, attached to a piece of clothing, head-mounted on the PPE, etc. In some aspects, the UV lamp 106 outputs light having UV-C wavelength (approximately 200-280 nanometers (nm)) but not UV-A wavelength (approximately 320-400 nm) and not UV-B wavelength (approximately 280-320 nm). The UV lamp 106 may output constant or variable intensity UV light, where the intensity of the UV-C light is generally controlled to be favorable for bacterial/viral disinfection applications while optimizing for user safety. It will be appreciated that UV-C light, such as “far” UV-C light having a wavelength of between 207-222 nm, may have the advantage of being relatively safe if the user's skin or eye is exposed to the light while still being able to kill or inactivate bacteria and viruses. Moreover, the dose of far UV-C light to kill or inactivate the bacteria or viruses may be relatively small (e.g., 2 millijoules (mJ) per square centimeter (cm²)). In a particular aspect, for added safety, the PPE worn by the user may comply with the American National Standards Institute's Z81 (“ANSI-Z81”) standard, protecting the user's face from the UV-C light.

In the same or an alternative implementation, the disinfection device 104 can include at least one of a chemical emitter 108, an aerosol emitter 110, an ultrasonic speaker 112, a microwave energy emitter 114, a robotic device, or other mechanical, electrical, and/or electromechanical device configured to initiate, perform, or otherwise address a pathogen disinfection operation. For example, the chemical emitter 108 can emit an antibacterial and/or antiviral chemical onto an infected or potentially infected surface or object. As an additional example, the aerosol emitter 110 can emit one or more disinfecting agents via an aerosol spray onto an infected or potentially infected surface or object. As an additional example, the ultrasonic speaker 112 can generate and/or direct ultrasonic sound waves to cause ultrasonic cavitation in a fluid (e.g., 70% isopropyl alcohol) for disinfection. As an additional example, the microwave energy emitter 114 can generate and/or direct microwave energy onto an infected or potentially infected surface or object. As an additional example, the robotic device can perform any number of actions directed toward disinfecting a surface or object, including cleaning, applying disinfectant materials, localized destruction of a portion of an infected surface or object, movement of a surface or object to another location, etc.

In some implementations, the system 100 also includes an input device 120 configured to receive input from the person wearing the PPE(s). The input device 120 can include one or more components configured to receive input from the person wearing the PPE(s). For example, the input device 120 can include one or more microphones 122 and/or one or more microphone arrays configured to receive user input 128 in the form of audio input (analog, digital, spoken, recorded, etc.). As an additional example, the input device 120 can include one or more network interfaces 124 configured to receive user input 128 via a network (e.g., the internet) from an external device (e.g., a smartphone, tablet, etc.). As yet another example, the input device 120 can include one or more tactile input devices 126 configured to receive user input 128 through a touch-based interaction between the user and the input device 120. The tactile input device 126 can include a button, touchpad, touch screen, etc.

In some implementations, the system 100 can also be configured to provide user output 130 via one or more output devices 132 configured to communicate certain information to the person wearing the PPE(s). For example, the output device 132 can include one or more audio devices 134 and/or one or more display devices 136. The audio device(s) 134 can include, for example, one or more speakers or speaker components configured to output audio information to a person wearing the PPE(s). In the same or alternative implementations, the output device(s) 132 can include one or more display devices 136 configured to output visual information to a person wearing the PPE(s). In a particular implementation, at least one of the display devices 136 is configured to display an augmented reality (“AR”) heads-up display (“HUD”) to the user of the PPE(s).

In some implementations, the audio information and/or the visual information can include instructions to the user on how to perform one or more disinfection operations. In a particular implementation, the output device 132 can be configured to output instructions to the user wearing the PPE(s) in order to walk the user through some or all of a disinfection procedure. For example, the audio device 134 may output and/or the display device 136 may display a first instruction 138 to place an object (e.g., a surface or object to be disinfected) or a body part (e.g., the user's gloved or ungloved hands) within a field of the disinfection device 104 (e.g., a UV lamp). The audio device 134 may output and/or the display device 136 may display a second instruction 140 to remove the object or body part from within field of the disinfection device 104 (e.g., a UV lamp). The audio device 134 may output and/or the display device 136 may display a third instruction 142 to move (e.g., rotate or reposition) the object or body part while the object or the body part is in the field of operation of the disinfection device 104. As another example, the audio device 134 may output and/or the display device 136 may display information regarding the appropriate location to begin a disinfection procedure.

In the same or alternative implementations, the audio device 134 may output, and/or the display device 136 may display output information regarding a power supply status 144 (e.g., a battery charge level, etc.) for the input device 120, the output device 132, and/or the disinfection device 104; a disinfection device status 146 of the disinfection device 104 (e.g., a decontaminant storage level, etc.); a PPE status 148 of the PPE(s) (e.g., a filter status, wear status, etc.), status of another components of the system 100, and/or some combination thereof.

In some implementations, some or all of the output device(s) 132 can be incorporated into one or more PPEs. For example, in a particular configuration in which the display device 136 of the output device 132 includes an AR HUD, the HUD can be external to one or more of the PPEs (e.g., the HUD can be a distinct AR headset worn apart from the PPE(s)). The HUD can also be wholly or partially incorporated into the PPE(s), disposed within the PPE(s), or some combination thereof. For example, the HUD can be configured to be displayed on an interior surface of a facemask covering a portion of the user's face, as described in more detail below with reference to FIG. 5.

In some implementations, the user output 130 provided by the output device 132 may be based on output data 150 communicated to the output device 132 from a computing device 102 communicatively coupled to the output device 132. The computing device 102 can include, in some implementations, one or more processors 118 communicatively coupled to a memory 116. In some implementations, the memory 116 includes volatile memory devices, non-volatile memory devices, or both, such as one or more hard drives, solid-state storage devices (e.g., flash memory, magnetic memory, or phase change memory), a random access memory (“RAM”), a read-only memory (“ROM”), one or more other types of storage devices, or any combination thereof. The memory 116 can be configured to store, as an illustrative example, the first, second, and third instructions 138-142 used by the output device 132 to walk a user through a disinfection procedure. As another illustrative example, the memory 116 can be configured to store the power supply status 144, the disinfection device status 146, the PPE status 148, and/or some combination thereof to be communicated to the output device 132 for communicating as the user output 130. The memory 116 can also be configured to store instructions that, when executed by the processor(s) 118, cause the processor(s) 118 to perform various functions with respect to the input device(s) 120, the output device(s) 132, and/or the disinfection device(s) 104, as described in more detail below and with reference to FIGS. 2-8. The processor(s) 118 include one or more single-core or multi-core processing units, one or more digital signal processors (DSPs), one or more graphics processing units (GPUs), or any combination thereof.

In a particular implementation, the input device(s) 120 can be configured to convert some or all of the user input 128 into input data 152 for communication to the computing device 102. Based on the input data 152, the processor(s) can be configured to determine how to respond to the user input 128 based on an analysis of the input data 152. For example, in certain configurations where the user provides speech input, the processor(s) 118 can be configured to execute one or more natural language processing (“NLP”) models to analyze the user's speech and determine what the user is saying and how to respond to the user's speech (e.g., “turn on lamp,” “turn off lamp,” “battery status,” “date check,” “time check,” “alert teammate,” “how much longer until disinfection is complete,” etc.). The analysis of the input data 152 can result in, among other actions, communicating output data 150 to the output device 132 for communication to the user as user output 130. For example, in response to user input of “battery status,” the computing device 102 can communicate the power supply status 144 as part of the output data 150 for communication to the user by the output device 132.

In a particular implementation, the processor(s) 118 can be configured to selectively activate and deactivate the disinfection device(s) 104 responsive to the user input 128 (e.g., as received by the microphone 122 and/or the tactile input device 126 of the input device 120). In some implementations, the selective activation can be accomplished through the communication of activation data 168 from the computing device 102 to the disinfection device(s) 104. For example, the processor(s) 118 can be configured to receive input data 152 associated with a user input 128 to activate the disinfection device(s) 104 as part of a disinfection procedure. The computing device 102 can then communicate the activation data 168 to the disinfection device(s) 104 responsive to receipt of the input data 152. The activation data 168 can include, for example, data indicative of a particular type of disinfection (e.g., ultraviolet, chemical, ultrasonic, microwave, etc.), instructions for a robotic component of the disinfection device(s) 104, a power on/off signal for the disinfection device(s) 104, a power duration signal for the disinfection device(s) 104, etc.

In some implementations, the activation data 168 can be based on a more complex analysis of data input to the computing device 102. For example, the computing device 102 can apply one or more machine learning models to the input data 152 in order to generate the activation data 168. To accomplish this in a particular implementation, the system 100 can include one or more sensors 154. The sensor(s) can include, for example, a thermal sensor, infrared sensor, biosensor, laboratory on-chip sensor, airborne particle analysis sensor, etc. Sensor output data 156 associated with one or more sensor readings by the sensor(s) 154 can be communicated from the sensor(s) 154 to the computing device 102. In a particular implementation, the processor(s) 118 can be configured to determine, based at least in part on the sensor output data 156, a likelihood that a particular environment of the person wearing the PPE(s) (and/or a particular surface or object within that environment) is infected by a pathogen. In a particular implementation, the processor(s) 118 can be further configured to selectively activate the disinfection device(s) 104 based at least in part on the likelihood of infection.

As an illustrative example, the processor(s) 118 can be configured to provide the sensor output data 156 as input to one or more infection likelihood models 158. The infection likelihood model(s) 158 may be machine learning models configured to generate an infection likelihood 160, as described in more detail below with reference to FIG. 2. The one or more infection likelihood models 158 can include an infection detection model, an alert generation model, or both.

In some implementations, the processor(s) 118 can be configured to select an infection likelihood model 158 from among a plurality of infection likelihood models. In a particular aspect, each of the plurality of infection likelihood models can be associated with a particular type or mode of sensor output analysis (e.g., infection detection, object identification, etc.). In the same or another particular aspect, each of the plurality of trained behavior models can be associated with one or more of a plurality of sensors 154 and/or one or more of the disinfection devices 104.

The processor(s) 118 can be configured to receive a portion of the sensor output data 156 during a sensing period. In some implementations, the one or more processors 118 are configured to process the portion of the sensor output data 156 to generate input data for the one or more infection likelihood models 158 and to use the one or more infection likelihood models 158 to generate the infection likelihood 160 for use in determining, via a likelihood output module 162, likelihood information 166 and/or determining, via a selective activation module 164, the activation data 168 for communication to the disinfection device(s) 104. The one or more processors 118 can also be configured to process the sensor output data 156 to determine whether to generate an alert.

In a particular aspect, the computing device 102 can be configured to receive the sensor output data 156 via a direct communication interface between the computing device 102 and the sensor(s) 154. In other particular aspects, the computing device 102 can be configured to receive the sensor output data 156 via one or more direct and/or indirect communication paths, including wired and/or wireless communication connection(s). In some implementations, the sensor(s) 154 send all or a portion of the sensor output data 156 to the computing device 102 in real time (e.g., while the sensor(s) 154 are still gathering data). In some implementations, the sensor(s) 154 gather and store the sensor output data 156 for later transmission to the computing device 102.

In some implementations, each of the sensors 154 can generate a time series of measurements. The time series from a particular sensor is also referred to herein as a “feature” or as “feature data.” Different sensors can have different sample rates. The sensor(s) 154 can generate sensor data samples periodically (e.g., with regularly spaced sampling periods). The sensor(s) 154 can also, or alternatively, generate sensor data samples occasionally (e.g., whenever a state change occurs).

During operation, the sensor(s) 154 can generate signals based on measuring physical characteristics, electromagnetic characteristics, radiologic characteristics, and/or other measurable characteristics associated with a potentially infected surface, object, and/or environment. In some implementations, the sensor(s) 154 can sample and encode (e.g., according to a communication protocol) the signals to generate the sensor output data 156. In some implementations, the sensor(s) 154 process the incoming sensor data to generate the sensor output data 156. For example, the sensor(s) 154 may calculate values of the sensor output data 156 from two or more sensors of the sensors 154. To illustrate, a first sensor may include an image sensor, and a second sensor may include a thermal sensor. In this illustrative example, the sensor output data 156 may include images from the first sensor, thermal readings from the second sensor, and/or a combination thereof. As another illustrative example, a first sensor may generate time domain signals and the first sensor or a second sensor may generate the sensor output data 156 by sampling and windowing the time domain signals and transforming windowed samples of the signal to a frequency domain. In still other implementations, the sampling, compressing, and/or other processing of sensor data may be accomplished by another processing unit coupled between the sensor(s) 154 and the computing device 102, by the computing device 102, or some combination thereof.

In some implementations, the processor(s) 118 receive some or all of the sensor output data 156 for a particular timeframe. During some timeframes, the sensor output data 156 for a particular timeframe may include a single data sample for each feature. During some timeframes, the sensor output data 156 for the particular timeframe may include multiple data samples for one or more of the features. During some timeframes, the sensor output data 156 for the particular timeframe may include no data samples for one or more of the features. As one example, if a first sensor registers state changes (e.g., on/off state changes), a second sensor generates a data sample once per second, a third sensor generates ten data samples per second, and the processor(s) 118 process one second timeframes, then for a particular timeframe the processor(s) 118 can receive sensor output data 156 that includes no data samples from the first sensor (e.g., if no state change occurred), one data sample from the second sensor, and ten samples from the third sensor. Other combinations of sampling rates and preprocessing timeframes are used in other examples.

In some implementations, the computing device 102 can include a preprocessor configured to generate the input data for the one or more infection likelihood models 158 based on the sensor output data 156. For example, the preprocessor can be configured to perform a batch normalization process on a portion of the sensor output data 156. As another example, the preprocessor may resample the sensor output data 156, may filter the sensor output data 156, may impute data, may use the sensor data (and possibly other data) to generate new feature data values, may perform other preprocessing operations, or a combination thereof. In a particular aspect, the specific preprocessing operations that a preprocessor performs can be determined based on the training of the one or more infection likelihood models 158. For example, an infection detection model can be trained to accept as input a specific set of features, and the preprocessor can be configured to generate, based on the sensor output data 156, input data for the infection detection model(s) including a specific set of features.

In a particular aspect, one or more of the infection likelihood models 158 (e.g., one or more infection detection models) can be configured to generate an infection likelihood 160 for each data sample of the input data. One or more of the infection detection models can be configured to evaluate the infection likelihood 160 to determine whether to generate an alert. As one example, an alert generation model can compare one or more values of the infection likelihood 160 to one or more respective thresholds to determine whether to generate an alert. The respective threshold(s) may be preconfigured or determined dynamically (e.g., based on one or more of the sensor data values, based on one or more of the input data values, or based on one or more of the infection likelihood 160 values). In a particular implementation, an alert generation model can be configured to determine whether to generate the alert using a sequential probability ratio test (SPRT) based on current infection likelihood 160 values and historical infection likelihood values (e.g., based on historical sensor data).

Thus, the system 100 can be configured to enable detection of deviation from a non-infected environment, such as detecting a transition from a first operating state (e.g., a “normal” state to which the model is trained) to a second operating state (e.g., an “abnormal” state). In some implementations, the second operating state, although distinct from the first operating state, may also be a “normal” operating state that is not associated with an infection or environmental condition in need of remediation.

Although certain illustrative examples are provided above for the infection likelihood model(s) 158, other types of infection likelihood model(s) 158 can be used without departing from the scope of the present disclosure. For example, the infection likelihood model 158 can include a dimensional-reduction model such as an autoencoder, a residual generator, an operation state classifier, or other appropriate type of trained behavior model.

In some implementations, the computing device can be configured to selectively activate the disinfection device(s) 104 based at least in part on the sensor output data 156. As described above, the activation data 168 can be used to, for example, selectively activate some or all of the disinfection device(s) 104. The infection likelihood 160 can be used by the selective activation module 164 to determine whether to selectively activate one or more of the disinfection device(s) 104. For example, the selective activation module 164 can compare the infection likelihood 160 to historical infection likelihood values as described above to determine whether the infection likelihood 160 meets a particular threshold. As an additional example, the selective activation module 164 can generate the activation data 168 if the infection likelihood 160 is above a particular threshold (e.g., if the infection likelihood is greater than 75%).

In the same or alternative implementations, the processor(s) 118 can be configured to perform certain analytical techniques to determine whether to selectively activate the disinfection device(s) 104 without determining whether or what particular pathogen(s) are or may be present on a surface and/or object. To illustrate, a forensic light source (“FLS”) similar to those used in crime scene investigation may be attached to the PPE(s) or otherwise carried by the user. Light from the FLS may be used to illuminate a surface and/or object, and the processor(s) 118 can employ a computer vision model operating on images captured by an optical sensor of the PPE to determine whether a potentially harmful substance (e.g., bodily fluids and/or droplets such as respiratory droplets, bodily fluids, etc.) is present on the surface. If so, the processor(s) 118 can generate activation data 168 to selectively activate the disinfection device(s) 104 without actually identifying whether/what pathogens may be present.

It should be understood that although some examples are described herein with reference to a predicted likelihood of infection, in alternative aspects a calculated likelihood of infection may be used instead of or in addition to a predicted likelihood.

In some implementations, a likelihood output module 162 of the processor(s) 118 can be configured to generate likelihood information 166 based at least on the infection likelihood 160 for communication to the output device 132. For example, the likelihood output module 162 can be configured to generate a message (e.g., “This area is likely contaminated,” “This object requires decontamination,” etc.) for output as likelihood information 166 to the output device 132. The output device 132 can be further configured to output the likelihood information 166 to the user wearing the PPE(s). For example, the audio device 134 of the output device 132 can play the likelihood information 166 aloud so that the user can hear it. As an additional example, the display device 136 of the output device 132 can display the likelihood information 166 (e.g., on the AR HUD) so that the user can view the likelihood information 166.

In a particular aspect, some or all of the likelihood information 166 can be communicated to the output device 132 via a direct communication interface between the computing device 102 and the output device 132. In other particular aspects, some or all of the likelihood information 166 can be communicated to the output device 132 via one or more direct and/or indirect communication paths, including a wired and/or wireless communication connection.

As an illustrative example of the system 100 in operation, a doctor, nurse, lab worker, infectious disease researcher, etc. may utilize the system 100 by wearing the PPE(s) and wearing and/or carrying the disinfection device(s) 104. The wearer may interact with the system using speech, tactile, and/or other input to get status information, selectively activate the disinfection device(s) 104, etc. One or more sensors 154 may interface with the PPE(s) (e.g., via the computing device 102), and in some cases the native spatial functionality of PPE headwear may be used within a headset to overlay sensor data from the individual sensors on the AR HUD. The user may thus be able to see what the sensors 154 are “picking up.” Different modes may be programmed to highlight specific things. For example, by setting thresholds on various sensor inputs, the user may see alerts. To illustrate, an alert may indicate that a temperature of a nearby face exceeds a certain temperature threshold. In this scenario a computer vision machine learning model may operate on the output of an optical sensor to detect a person's face and the thermal sensor to indicate a high temperature. As yet another example, a computer vision machine learning model may be used to automatically identify objects or surfaces that are often touched, such as doorknobs, chair handles, light switches, etc. When such objects are identified, the UV lamp may automatically be activated to disinfect such objects. As the user moves around an area, different combinations of readings and/or objects may be detected, and different instructions may be provided to the user. The instructions could be to clean or disinfect a certain area, avoid a certain area, treat a certain person, etc. Moreover, the HUD may notify the user when a disinfectant has been applied long enough to a contaminated object and/or surface (e.g., based on when a timer has elapsed, based on spectral analysis of the surface based on images/video of the surface captured by sensors, etc.).

Although FIG. 1 illustrates certain components arranged in a particular manner, more, fewer, and/or different components can be present without departing from the scope of the present disclosure. For example, FIG. 1 illustrates the processor(s) 118 and the memory 116 within the computing device 102. In some implementations, the processor(s) 118 and/or the memory 116 can instead be located (either co-located or distributed) in or among other components of the system 100. For example, the processor(s) 118 and the memory 116 may be located within the disinfection device 104. As an additional example, the processor(s) 118 and the memory 116 can be located within the display device 136 of the output device 132 (e.g., as part of a VR headset).

FIG. 2 depicts a block diagram of a particular implementation of components that may be included in the system 100 of FIG. 1 in accordance with some examples of the present disclosure. The block diagram 200 illustrates components that can be configured to provide, as input to one or more infection likelihood models 158, input data to generate the alert 228.

As illustrated, the infection detection model 202 includes one or more infection likelihood models 158, a residual generator 204, and an infection likelihood calculator 206. The one or more infection likelihood models 158 include an autoencoder 210, a time series predictor 212, a feature predictor 214, another behavior model, or a combination thereof. Each of the infection likelihood model(s) 158 is trained to receive sensor output data 156 (e.g., from the processor(s) 118) and to generate a model output. The residual generator 204 is configured to compare one or more values of the model output to one or more values of the sensor output data 156 to determine the residuals data 208.

The autoencoder 210 may include or correspond to a dimensional-reduction type autoencoder, a denoising autoencoder, or a sparse autoencoder. Additionally, in some implementations the autoencoder 210 has a symmetric architecture (e.g., an encoder portion of the autoencoder 210 and a decoder portion of the autoencoder 210 have mirror-image architectures). In other implementations, the autoencoder 210 has a non-symmetric architecture (e.g., the encoder portion has a different number, type, size, or arrangement of layers than the decoder portion).

The autoencoder 210 is trained to receive model input (denoted as zₜ), modify the model input, and reconstruct the model input to generate model output (denoted as z′ₜ). The model input includes values of one or more features of the sensor output data 156 (e.g., raw and/or preprocessed readings from one or more sensors) for a particular timeframe (t), and the model output includes estimated values of the one or more features (e.g., the same features as the model input) for the particular timeframe (t) (e.g., the same timeframe as the model input). In a particular, non-limiting example, the autoencoder 210 is an unsupervised neural network that includes an encoder portion to compress the model input to a latent space (e.g., a layer that contains a compressed representation of the model input), and a decoder portion to reconstruct the model input from the latent space to generate the model output. The autoencoder 210 can be generated and/or trained via an automated model building process, an optimization process, or a combination thereof to reduce or minimize a reconstruction error between the model input (zₜ) and the model output (z′ₜ) when the sensor output data 156 represents normal operation conditions associated with a monitored environment.

The time series predictor 212 may include or correspond to one or more neural networks trained to forecast future data values (such as a regression model or a generative model). The time series predictor 212 is trained to receive as model input one or more values of the sensor output data 156 (denoted as zₜ) for a particular timeframe (t) and to estimate or predict one or more values of the sensor output data 156 for a future timeframe (t+1) to generate model output (denoted as z′ₜ+1). The model input includes values of one or more features of the sensor output data 156 (e.g., readings from one or more sensors) for the particular timeframe (t), and the model output includes estimated values of the one or more features (e.g., the same features at the model input) for a different timeframe (t+1) than the timeframe of the model input. The time series predictor 212 can be generated and/or trained via an automated model building process, an optimization process, or a combination thereof, to reduce or minimize a prediction error between the model input (zₜ) and the model output (z′t+1) when the sensor output data 156 represents normal operation conditions associated with a monitored environment.

The feature predictor 214 may include or correspond to one or more neural networks trained to predict data values based on other data values (such as a regression model or a generative model). The feature predictor 214 is trained to receive as model input one or more values of the sensor output data 156 (denoted as zₜ) for a particular timeframe (t) and to estimate or predict one or more other values of the sensor output data 156 (denoted as yₜ) to generate model output (denoted as y′ₜ). The model input includes values of one or more features of the sensor output data 156 (e.g., readings from one or more sensors) for the particular timeframe (t), and the model output includes estimated values of the one or more other features of the sensor output data 156 for the particular timeframe (t) (e.g., the same timeframe as the model input). The feature predictor 214 can be generated and/or trained via an automated model building process, an optimization process, or a combination thereof, to reduce or minimize a prediction error between the model input (zₜ) and the model output (y′ₜ) when the sensor output data 156 represents normal operation conditions associated with a monitored environment.

In certain implementations, the infection detection model 202 can use one or more of the infection likelihood models 158 according to the one or more model selection criteria, as described above with reference to FIG. 1. In some aspects, the infection detection model 202 can use one or more behavior models of one or more behavior model types (e.g., one or more autoencoders 210, one or more time series predictors 212, one or more feature predictors 214, or some combination thereof). The model selection criteria can be used to identify the infection likelihood model(s) 158 to be used by the infection detection model 202.

The residual generator 204 is configured to generate a residual value (denoted as r) based on a difference between the model output of the infection likelihood model(s) 158 and the sensor output data 156. For example, when the model output is generated by an autoencoder 210, the residual can be determined according to r=z′ₜ−zₜ. As another example, when the model output is generated by a time series predictor 212, the residual can be determined according to r=z′ₜ+1−zₜ+1, where z′ₜ+1 is estimated based on data for a prior time step (t) and z′ₜ+1 is the actual value of z for a later time step (t+1). As still another example, when the model output is generated by a feature predictor 214, the residual can be determined according to r=y′ₜ−yₜ, where y′ₜ is estimated based on a value of z for a particular time step (t) and yₜ is the actual value of y for the particular time step (t). Generally, the sensor output data 156 and the reconstruction are multivariate (e.g., a set of multiple values, with each value representing a feature of the sensor output data 156), in which case multiple residuals are generated for each sample time frame to form the residuals data 208 for the sample time frame.

The infection likelihood calculator 206 determines the infection likelihood 160 for a sample time frame based on the residuals data 208. The infection likelihood 160 is provided to the alert generation model 218. The alert generation model 218 evaluates the infection likelihood 160 to determine whether to generate the alert 228. As one example, the alert generation model 218 compares one or more values of the infection likelihood 160 to one or more respective thresholds to determine whether to generate the alert 228. The respective threshold(s) may be preconfigured or determined dynamically (e.g., based on one or more of the sensor data values, based on one or more of the input data values, or based on one or more of the values of the infection likelihood 160).

In a particular implementation, the alert generation model 218 determines whether to generate the alert 228 using a sequential probability ratio test (SPRT) based on current infection likelihood 160 values and historical infection likelihood 160 values (e.g., based on historical sensor data). In FIG. 2, the alert generation model 218 accumulates a set of infection scores 220 representing multiple sample time frames and uses the set of infection scores 220 to generate statistical data 222. In the illustrated example, the alert generation model 218 uses the statistical data 222 to perform a sequential probability ratio test 224 configured to selectively generate the alert 228. For example, the sequential probability ratio test 224 is a sequential hypothesis test that provides continuous validations or refutations of the hypothesis that the monitored asset is behaving abnormally, by determining whether the infection likelihood 160 continues to follow, or no longer follows, normal behavior statistics in view of reference infection scores 226. In some implementations, the reference infection scores 226 include data indicative of a distribution of reference infection scores (e.g., mean and variance) instead of, or in addition to, the actual values of the reference infection scores. The sequential probability ratio test 224 provides an early detection mechanism and supports tolerance specifications for false positives and false negatives.

In some implementations, the alert 228 generated by the alert generation model 218 can be communicated to a likelihood output module such as the likelihood output module 162 of FIG. 1. The likelihood output module can be configured to generate the likelihood information 166 for communication to the output device 132. The likelihood information 166 can include, for example, data indicative of a message instructing a user that there is a high likelihood of infection within a monitored environment as indicated by the alert 228.

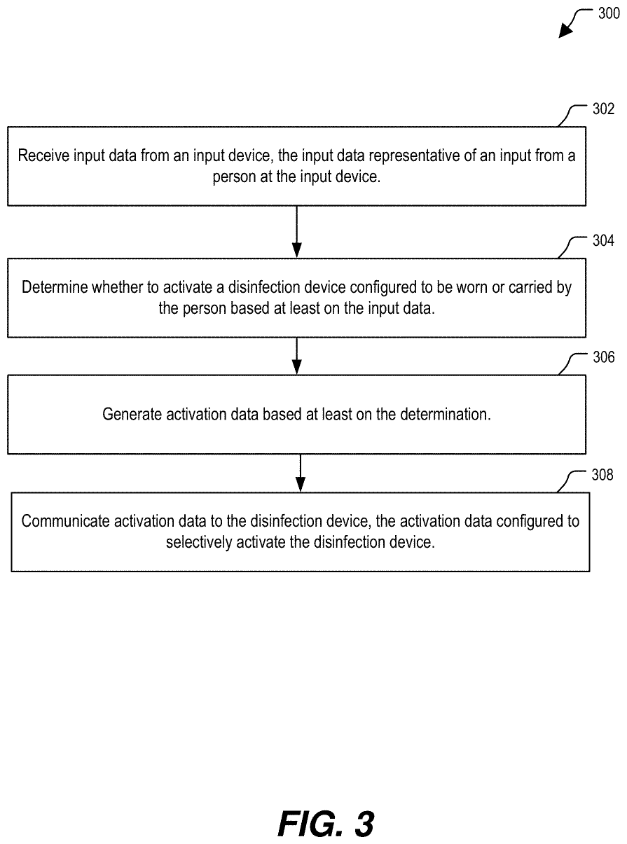

FIG. 3 is a flow chart of an example of a method 300 for personal protection and pathogen disinfection, in accordance with some examples of the present disclosure. The method 300 may be initiated, performed, or controlled by one or more processors executing instructions, such as by the processor(s) 118 of FIG. 1 executing instructions such as instructions from the memory 116.

In some implementations, the method 300 includes, at 302, receiving input data from an input device, the input data representative of an input from a person at the input device. For example, as described in more detail above with reference to FIGS. 1-2, the input device 120 can communicate the input data 152 to the computing device 102, wherein the input data 152 is representative of the user input 128.

In the example of FIG. 3, the method 300 also includes, at 304, determining whether to activate a disinfection device configured to be worn or carried by the person based at least on the input data. For example, as described in more detail above with reference to FIGS. 1-2, the processor(s) 118 can be configured to determine whether to selectively activate one or more disinfection devices 104 based at least on the input data 152.

In the example of FIG. 3, the method 300 also includes, at 306, generating activation data based at least on the determination. For example, as described in more detail above with reference to FIGS. 1-2, the processor(s) 118 can be configured to generate the activation data 168 based at least on determining whether to activate the one or more disinfection devices 104. In a particular aspect, generating the activation data 168 can include generating the activation data 168 based at least in part on the sensor output data 156 of the sensor(s) 154.

In the example of FIG. 3, the method 300 also includes, at 308, communicating activation data to the disinfection device, the activation data configured to selectively activate the disinfection device. For example, as described in more detail above with reference to FIGS. 1-2, the processor(s) 118 can be configured to communicate the activation data 168 to the disinfection device(s) 104, wherein the activation data 168 is configured to selectively activate the disinfection device(s) 104.

Although the method 300 is illustrated as including a certain number of steps, more, fewer, and/or different steps can be included in the method 300 without departing from the scope of the present disclosure. For example, the method 300 can also include preprocessing sensor data prior to providing the sensor output data 156 as input to the infection likelihood model(s) 158 and communicating the preprocessed sensor data to the processor(s) 118. As an additional example, the method 300 can also include communicating information to the person via an output device and/or determining, based at least in part on an output of the sensor, a likelihood that a particular object or environment of the person is infected by a pathogen.

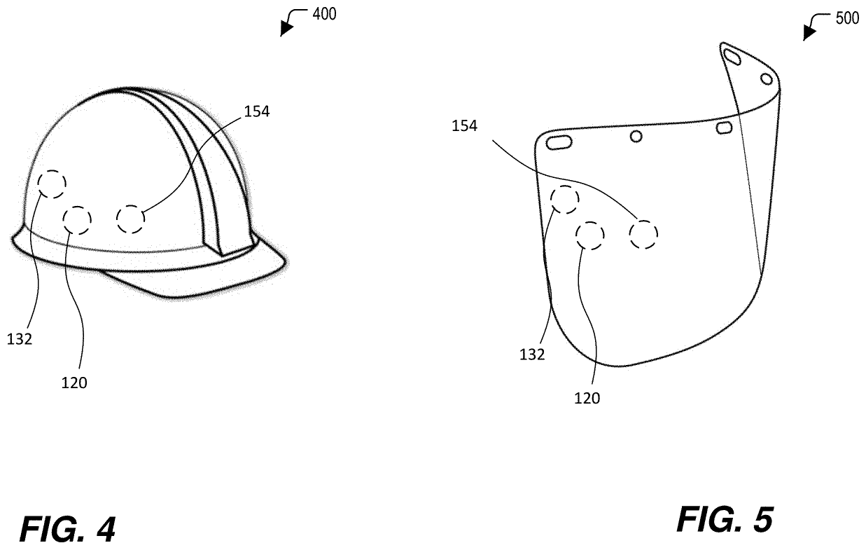

FIG. 4 is an illustrative example of a PPE including a helmet 400 that incorporates aspects of the system 100 of FIG. 1. In FIG. 4, the helmet 400 can include one or more components of the output device 132, one or more components of the input device 120, and/or one or more sensors 154. In a particular implementation, the input device 120 can include one or more microphones (e.g., the microphone 122 of FIG. 1), as described in more detail above with reference to FIG. 1. In the same or alternative particular implementations, the output device 132 can include one or more speakers (e.g., the audio device 134 of FIG. 1), as described in more detail above with reference to FIG. 1. Thus, the techniques described with respect to FIGS. 1-3 enable the aspects of the system 100 coupled to a PPE including the helmet 400 to protect the user of the PPE.

FIG. 5 is an illustrative example of a PPE including a face shield 500 that incorporates aspects of the system 100 of FIG. 1. In FIG. 5, the face shield 500 can include one or more components of the output device 132, one or more components of the input device 120, and/or one or more sensors 154. In a particular implementation, the input device 120 can include one or more microphones (e.g., the microphone 122 of FIG. 1), as described in more detail above with reference to FIG. 1. In the same or alternative particular implementations, the output device 132 can include one or more speakers (e.g., the audio device 134 of FIG. 1), as described in more detail above with reference to FIG. 1. Thus, the techniques described with respect to FIGS. 1-3 enable the aspects of the system 100 coupled to a PPE including the face shield 500 to protect the user of the PPE.

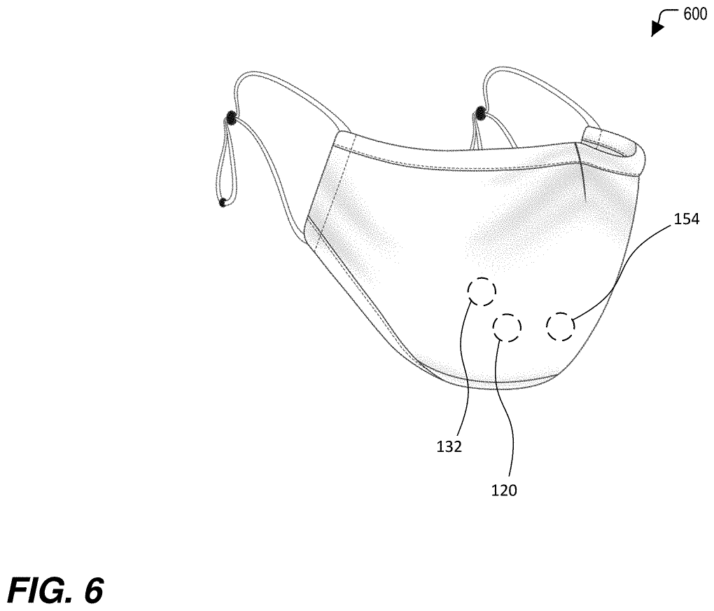

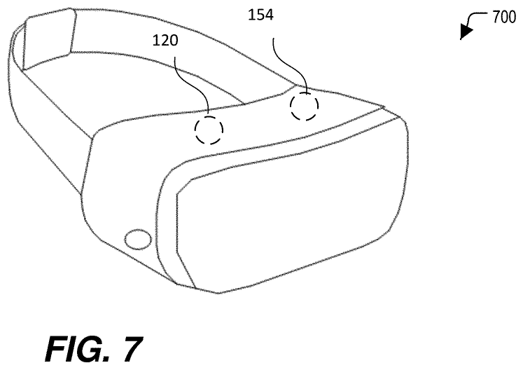

FIG. 6 is an illustrative example of a PPE including a mask 600 that incorporates aspects of the system 100 of FIG. 1. In FIG. 6, the mask 600 can include one or more components of the output device 132, one or more components of the input device 120, and/or one or more sensors 154. In a particular implementation, the input device 120 can include one or more microphones (e.g., the microphone 122 of FIG. 1), as described in more detail above with reference to FIG. 1. In the same or alternative particular implementations, the output device 132 can include one or more speakers (e.g., the audio device 134 of FIG. 1), as described in more detail above with reference to FIG. 1. Thus, the techniques described with respect to FIGS. 1-3 enable the aspects of the system 100 coupled to a PPE including the mask 600 to protect the user of the PPE. Although FIG. 6 illustrates certain aspects of a PPE as a mask 600, aspects of the system 100 of FIG. 1 can likewise be incorporated into a mask cover in a similar manner without departing from the scope of the present disclosure.

FIG. 7 is an illustrative example of a headset 700 that incorporates certain aspects of the system 100 of FIG. 1. Generally, the headset 700 is an illustrative example of the display device 136 of FIG. 1 described in more detail above. In some implementations, the headset 700 may include other aspects of the system 100 of FIG. 1. For example, the headset 700 can include one or more components of the input device 120 and/or one or more sensors 154. In a particular implementation, the input device 120 can include one or more microphones (e.g., the microphone 122 of FIG. 1), as described in more detail above with reference to FIG. 1. In the same or alternative particular implementations, the headset 700 can include one or more speakers (e.g., the audio device 134 of FIG. 1), as described in more detail above with reference to FIG. 1. Thus, the techniques described with respect to FIGS. 1-3 enable the aspects of the system 100 embodied in the headset 700 to communicatively couple to one or more PPEs to protect the user of the PPE. Although FIG. 7 illustrates the headset 700 as external to the PPE(s), the headset can be part of the PPE(s), disposed within the PPE(s) (e.g., as part of the helmet 400 of FIG. 4, the face shield of FIG. 5, the mask 600 or mask cover of FIG. 6, etc.), or some combination thereof without departing from the scope of the present disclosure.

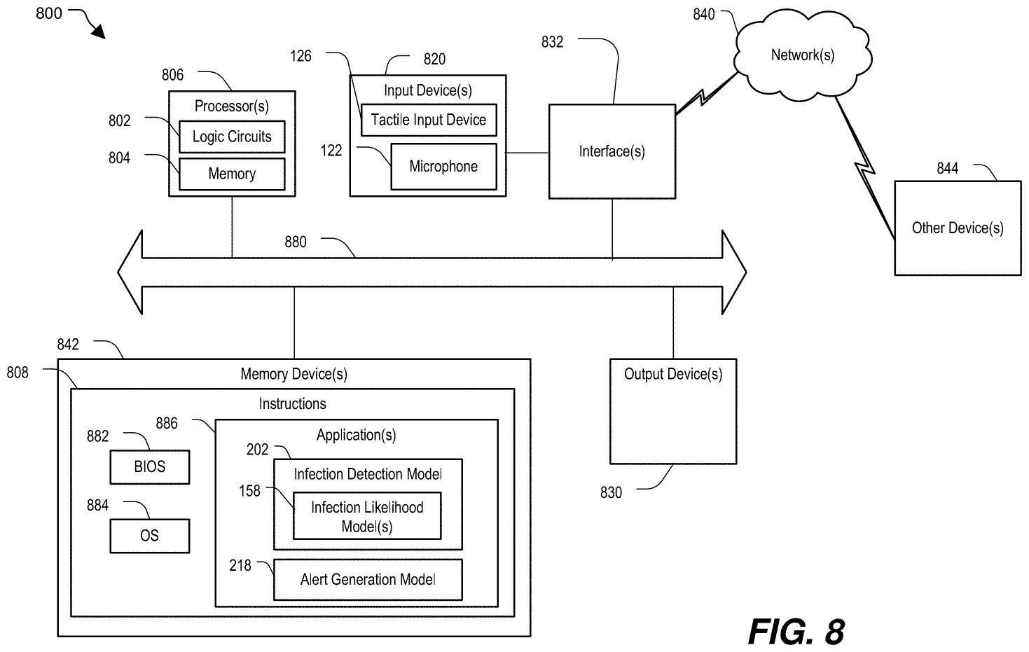

FIG. 8 illustrates an example of a computer system 800 corresponding to the system 100 of FIG. 1. The computer system 800 can correspond to, include, or be included within the system 100, including the computing device 102 of FIG. 1, the disinfection device 104, and/or the input device 120. For example, the computer system 800 is configured to initiate, perform, or control one or more of the operations described with reference to FIGS. 1-7. The computer system 800 can be implemented as or incorporated into one or more of various other devices, such as a personal computer (PC), a tablet PC, a server computer, a personal digital assistant (PDA), a laptop computer, a desktop computer, a communications device, a wireless telephone, or any other machine capable of executing a set of instructions (sequential or otherwise) that specify actions to be taken by that machine. Further, while a single computer system 800 is illustrated, the term “system” includes any collection of systems or sub-systems that individually or jointly execute a set, or multiple sets, of instructions to perform one or more computer functions.

While FIG. 8 illustrates one example of the computer system 800, other computer systems or computing architectures and configurations may be used for carrying out the personal protection and pathogen disinfection operations disclosed herein. The computer system 800 includes one or more processors 806. Each processor of the one or more processors 806 can include a single processing core or multiple processing cores that operate sequentially, in parallel, or sequentially at times and in parallel at other times. Each processor of the one or more processors 806 includes circuitry defining a plurality of logic circuits 802, working memory 804 (e.g., registers and cache memory), communication circuits, etc., which together enable the processor(s) 806 to control the operations performed by the computer system 800 and enable the processor(s) 806 to generate a useful result based on analysis of particular data and execution of specific instructions.

The processor(s) 806 are configured to interact with other components or subsystems of the computer system 800 via a bus 880. The bus 880 is illustrative of any interconnection scheme serving to link the subsystems of the computer system 800, external subsystems or devices, or any combination thereof. The bus 880 includes a plurality of conductors to facilitate communication of electrical and/or electromagnetic signals between the components or subsystems of the computer system 800. Additionally, the bus 880 includes one or more bus controllers or other circuits (e.g., transmitters and receivers) that manage signaling via the plurality of conductors and that cause signals sent via the plurality of conductors to conform to particular communication protocols.

The computer system 800 also includes the one or more memory devices 842. The memory device(s) 842 include any suitable computer-readable storage device depending on, for example, whether data access needs to be bi-directional or unidirectional, speed of data access required, memory capacity required, other factors related to data access, or any combination thereof. Generally, the memory device(s) 842 includes some combinations of volatile memory devices and non-volatile memory devices, though in some implementations, only one or the other may be present. Examples of volatile memory devices and circuits include registers, caches, latches, many types of random-access memory (RAM), such as dynamic random-access memory (DRAM), etc. Examples of non-volatile memory devices and circuits include hard disks, optical disks, flash memory, and certain type of RAM, such as resistive random-access memory (ReRAM). Other examples of both volatile and non-volatile memory devices can be used as well, or in the alternative, so long as such memory devices store information in a physical, tangible medium. Thus, the memory device(s) 842 include circuits and structures and are not merely signals or other transitory phenomena (i.e., are non-transitory media).

In the example illustrated in FIG. 8, the memory device(s) 842 store the instructions 808 that are executable by the processor(s) 806 to perform various operations and functions. The instructions 808 include instructions to enable the various components and subsystems of the computer system 800 to operate, interact with one another, and interact with a user, such as an input/output system (BIOS) 882 and an operating system (OS) 884. Additionally, the instructions 808 include one or more applications 886, scripts, or other program code to enable the processor(s) 806 to perform the operations described herein. The applications 886 can include, as illustrative examples, the infection detection model 202 and/or the alert generation model 218 of FIG. 2, one or more infection likelihood models 158 of FIG. 1, the likelihood output module 162 of FIG. 1, the selective activation module 164 of FIG. 1, or some combination thereof.

In FIG. 8, the computer system 800 also includes one or more output devices 830, one or more input devices 820, and one or more interface devices 832. Each of the output device(s) 830, the input device(s) 820, and the interface device(s) 832 can be coupled to the bus 880 via a port or connector, such as a Universal Serial Bus port, a digital visual interface (DVI) port, a serial ATA (SATA) port, a small computer system interface (SCSI) port, a high-definition media interface (HDMI) port, or another serial or parallel port. In some implementations, one or more of the output device(s) 830, the input device(s) 820, the interface device(s) 832 is coupled to or integrated within a housing with the processor(s) 806 and the memory device(s) 842, in which case the connections to the bus 880 can be internal, such as via an expansion slot or other card-to-card connector. In other implementations, the processor(s) 806 and the memory device(s) 842 are integrated within a housing that includes one or more external ports, and one or more of the output device(s) 830, the input device(s) 820, the interface device(s) 832 is coupled to the bus 880 via the external port(s).

Examples of the output device(s) 830 include display devices, speakers, printers, televisions, projectors, or other devices to provide output of data in a manner that is perceptible by a user. Examples of the input device(s) 820 include buttons, switches, knobs, a tactile input device 126, a microphone 122, the network interface 124 of FIG. 1, a keyboard, a pointing device, a biometric device, a microphone, a motion sensor, or another device to detect user input actions. The tactile input device 126 can include, for example, one or more of a stylus, a pen, a touch pad, a touch screen, a tablet, another device that is useful for interacting with a graphical user interface, or any combination thereof. A particular device may be an input device 820 and an output device 830. For example, the particular device may be a touch screen.

The interface device(s) 832 are configured to enable the computer system 800 to communicate with one or more other devices 844 directly or via one or more networks 840. For example, the interface device(s) 832 may encode data in electrical and/or electromagnetic signals that are transmitted to the other device(s) 844 as control signals or packet-based communication using pre-defined communication protocols. As another example, the interface device(s) 832 may receive and decode electrical and/or electromagnetic signals that are transmitted by the other device(s) 844. To illustrate, the other device(s) 844 may include the sensor(s) 154 of FIG. 1. The electrical and/or electromagnetic signals can be transmitted wirelessly (e.g., via propagation through free space), via one or more wires, cables, optical fibers, or via a combination of wired and wireless transmission.

In an alternative embodiment, dedicated hardware implementations, such as application specific integrated circuits, programmable logic arrays and other hardware devices, can be constructed to implement one or more of the operations described herein. Accordingly, the present disclosure encompasses software, firmware, and hardware implementations.

The systems and methods illustrated herein may be described in terms of functional block components, screen shots, optional selections, and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, Java, JavaScript, VBScript, Macromedia Cold Fusion, COBOL, Microsoft Active Server Pages, assembly, PERL, PHP, AWK, Python, Visual Basic, SQL Stored Procedures, PL/SQL, any UNIX shell script, and extensible markup language (XML) with the various algorithms being implemented with any combination of data structures, objects, processes, routines or other programming elements. Further, it should be noted that the system may employ any number of techniques for data transmission, signaling, data processing, network control, and the like.

The systems and methods of the present disclosure may be embodied as a customization of an existing system, an add-on product, a processing apparatus executing upgraded software, a standalone system, a distributed system, a method, a data processing system, a device for data processing, and/or a computer program product. Accordingly, any portion of the system or a module or a decision model may take the form of a processing apparatus executing code, an internet based (e.g., cloud computing) embodiment, an entirely hardware embodiment, or an embodiment combining aspects of the internet, software, and hardware. Furthermore, the system may take the form of a computer program product on a computer-readable storage medium or device having computer-readable program code (e.g., instructions) embodied or stored in the storage medium or device. Any suitable computer-readable storage medium or device may be utilized, including hard disks, CD-ROM, optical storage devices, magnetic storage devices, and/or other storage media. As used herein, a “computer-readable storage medium” or “computer-readable storage device” is not a signal.

Systems and methods may be described herein with reference to block diagrams and flowchart illustrations of methods, apparatuses (e.g., systems), and computer media according to various aspects. It will be understood that each functional block of a block diagram or flowchart illustration, and combinations of functional blocks in block diagrams and flowchart illustrations, respectively, can be implemented by computer program instructions.

Computer program instructions may be loaded onto a computer or other programmable data processing apparatus to produce a machine, such that the instructions that execute on the computer or other programmable data processing apparatus create means for implementing the functions specified in the flowchart block or blocks. These computer program instructions may also be stored in a computer-readable memory or device that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions.

In conjunction with the described devices and techniques, an apparatus for personal protection and pathogen disinfection is disclosed that can include means for receiving input data from an input device, the input data representative of an input from a person at the input device. For example, the means for receiving can correspond to the computing device 102 of FIG. 1, the processor(s) 118 of FIG. 1, the input device 120 of FIG. 1, one or more other circuits or devices to receive input data, or any combination thereof.

The apparatus can also include means for determining whether to activate a disinfection device configured to be worn or carried by the person based at least on the input data. For example, the means for determining can correspond to the computing device 102 of FIG. 1, the processor(s) 118 of FIG. 1, the disinfection device 104 of FIG. 1, the selective activation module 164 of FIG. 1, one or more other circuits or devices to determine whether to activate a disinfection device, or any combination thereof.

The apparatus can also include means for generating activation data based at least on the determination. For example, the means for generating can correspond to the computing device 102 of FIG. 1, the processor(s) 118 of FIG. 1, the disinfection device 104 of FIG. 1, the selective activation module 164 of FIG. 1, one or more other circuits or devices to generate activation data, or any combination thereof.

The apparatus can also include means for communicating activation data to the disinfection device, the activation data configured to selectively activate the disinfection device. For example, the means for communicating activation data can correspond to the computing device 102 of FIG. 1, the processor(s) 118 of FIG. 1, the disinfection device 104 of FIG. 1, one or more other circuits or devices to communicate activation data, or any combination thereof.

Particular aspects of the disclosure are described below in the following clauses:

According to Clause 1, a personal protection and pathogen disinfection system includes a personal protective equipment (PPE) configured to cover at least a portion of a person's face when worn by the person. The system also includes a disinfection device configured to be worn or carried by the person. The system also includes an input device configured to receive input from the person. The system also includes at least one processor configured to selectively activate the disinfection device responsive to the input.

Clause 2 includes the system of Clause 1, wherein the PPE includes a helmet.

Clause 3 includes the system of Clause 1 or Clause 2, wherein the PPE includes a mask or mask cover.

Clause 4 includes the system of any of Clauses 1-3, wherein the PPE includes a face shield.

Clause 5 includes the system of any of Clauses 1-4, wherein the PPE is compliant with an ANSI-Z81 standard.

Clause 6 includes the system of any of Clauses 1-5, wherein the input device includes at least one of a microphone or microphone array configured to receive speech input from the person or a tactile input device configured to receive tactile input from the person.

Clause 7 includes the system of any of Clauses 1-6, wherein the input device includes a network interface configured to receive input via a network.

Clause 8 includes the system of any of Clauses 1-7, wherein the system also includes an output device configured to communicate information to the person.

Clause 9 includes the system of Clause 8, wherein the output device includes an audio device.

Clause 10 includes the system of Clause 8 or Clause 9, wherein the output device includes a display device configured to display an augmented reality (AR) heads-up display (HUD).

Clause 11 includes the system of Clause 10, wherein the display device is external to the PPE.

Clause 12 includes the system of Clause 10, wherein the display device is part of the PPE, disposed within the PPE, or both.

Clause 13 includes the system of any of Clauses 1-12, wherein the disinfection device includes a lamp configured to output ultraviolet (UV) light.

Clause 14 includes the system of Clause 13, wherein the UV light includes UV-C light, does not include UV-A light, and does not include UV-B light.

Clause 15 includes the system of any of Clauses 1-15, wherein the disinfection device includes at least one of a chemical emitter, an aerosol emitter, an ultrasonic speaker, a microwave energy emitter, or a robotic device.

Clause 16 includes the system of any of Clauses 1-15, wherein the system also includes an output device configured to output at least one of: a first instruction to place an object or a body part within a field of operation of the disinfection device; a second instruction to remove the object or the body part from within the field of operation of the disinfection device; or a third instruction to move the object or the body part while the object or the body part is in the field of operation of the disinfection device.

Clause 17 includes the system of any of Clauses 1-16, wherein the system also includes an output device configured to output information regarding a status of at least one of a power supply, the disinfection device, or the PPE.

Clause 18 includes the system of any of Clauses 1-17, wherein the system also includes a sensor.

Clause 19 includes the system of Clause 18, wherein the processor is further configured to selectively activate the disinfection device based at least in part on an output of the sensor.

Clause 20 includes the system of Clause 18 or Clause 19, wherein the sensor includes at least one of a thermal sensor, an optical sensor, an infrared sensor, a biosensor, a lab-on-chip sensor, or an airborne particle analysis sensor.

Clause 21 includes the system of any of Clauses 18-20, wherein the processor is configured to determine, based at least in part on an output of the sensor, a likelihood that a particular object or environment of the person is infected by a pathogen.

Clause 22 includes the system of Clause 21, wherein the processor is further configured to selectively activate the disinfection device based at least in part on the likelihood.

Clause 23 includes the system of Clause 21 or Clause 22, wherein the system also includes an output device configured to output information based on the likelihood.

According to Clause 24, a method includes receiving input data from an input device, the input data representative of an input from a person at the input device; determining whether to activate a disinfection device configured to be worn or carried by the person based at least on the input data; generating activation data based at least on the determination; and communicating activation data to the disinfection device, the activation data configured to selectively activate the disinfection device.

Clause 25 includes the method of Clause 24, wherein the input device includes at least one of a microphone or microphone array configured to receive speech input from the person or a tactile input device configured to receive tactile input from the person.

Clause 26 includes the method of Clause 24 or Clause 25, wherein the input device includes a network interface configured to receive input via a network.

Clause 27 includes the method of any of Clauses 24-26, wherein the method also includes communicating information to the person via an output device.

Clause 28 includes the method of Clause 27, wherein the information includes at least one of: a first instruction to place an object or a body part within a field of operation of the disinfection device; a second instruction to remove the object or the body part from within the field of operation of the disinfection device; or a third instruction to move the object or the body part while the object or the body part is in the field of operation of the disinfection device.

Clause 29 includes the method of Clause 27 or Clause 28, wherein the information includes information regarding a status of at least one of a power supply, the disinfection device, or a personal protective equipment (PPE) configured to cover at least a portion of the person's face when worn by the person.

Clause 30 includes the method of any of Clauses 27-29, wherein the output device includes an audio device.

Clause 31 includes the method of any of Clause 27-30, wherein the output device includes a display device configured to display an augmented reality (AR) heads-up display (HUD).

Clause 32 includes the method of Clause 31, wherein the display device is external to a personal protective equipment (PPE) configured to cover at least a portion of the person's face when worn by the person.

Clause 33 includes the method of Clause 31, wherein the display device is part of a personal protective equipment (PPE) configured to cover at least a portion of the person's face when worn by the person, disposed within the PPE, or both.

Clause 34 includes the method of Clause 32 or Clause 33, wherein the PPE includes a helmet.

Clause 35 includes the method of any of Clauses 32-34, wherein the PPE includes a mask or mask cover.

Clause 36 includes the method of any of Clauses 32-35, wherein the PPE includes a face shield.

Clause 37 includes the method of any of Clauses 32-36, wherein the PPE is compliant with an American National Standards Institute (ANSI) Z81 standard.

Clause 38 includes the method of any of Clauses 24-37, wherein the disinfection device includes a lamp configured to output ultraviolet (UV) light.

Clause 39 includes the method of Clause 38, wherein the UV light includes UV-C light, does not include UV-A light, and does not include UV-B light.

Clause 40 includes the method of any of Clauses 24-39, wherein the disinfection device includes at least one of a chemical emitter, an aerosol emitter, an ultrasonic speaker, a microwave energy emitter, or a robotic device.

Clause 41 includes the method of any of Clauses 24-40, wherein determining whether to activate the disinfection device is further based at least in part on an output of a sensor.

Clause 42 includes the method of Clause 41, wherein the sensor includes at least one of a thermal sensor, an optical sensor, an infrared sensor, a biosensor, a lab-on-chip sensor, or an airborne particle analysis sensor.

Clause 43 includes the method of Clause 42, wherein the method also includes determining, based at least in part on an output of the sensor, a likelihood that a particular object or environment of the person is infected by a pathogen.

Clause 44 includes the method of Clause 43, wherein generating activation data is further based at least in part on the likelihood.

Clause 45 includes the method of Clause 44, wherein the method also includes communicating information to the person via an output device, the information based at least in part on the likelihood.

According to Clause 46, a computer-readable storage device stores instructions that, when executed by one or more processors, cause the one or more processors to receive input data from an input device, the input data representative of an input from a person at the input device; determine whether to activate a disinfection device configured to be worn or carried by the person based at least on the input data; generate activation data based at least on the determination; and communicate activation data to the disinfection device, the activation data configured to selectively activate the disinfection device.

Clause 47 includes the computer-readable storage device of Clause 46, wherein the input device includes at least one of a microphone or microphone array configured to receive speech input from the person or a tactile input device configured to receive tactile input from the person.

Clause 48 includes the computer-readable storage device of Clause 46 or Clause 47, wherein the input device includes a network interface configured to receive input via a network.

Clause 49 includes the computer-readable storage device of any of Clauses 46-48, wherein the instructions, when executed by the one or more processors, further cause the one or more processors to communicate information to the person via an output device.

Clause 50 includes the computer-readable storage device of Clause 49, wherein the information includes at least one of: a first instruction to place an object or a body part within a field of operation of the disinfection device; a second instruction to remove the object or the body part from within the field of operation of the disinfection device; or a third instruction to move the object or the body part while the object or the body part is in the field of operation of the disinfection device.

Clause 51 includes the computer-readable storage device of Clause 49 or Clause 50, wherein the information includes information regarding a status of at least one of a power supply, the disinfection device, or a personal protective equipment (PPE) configured to cover at least a portion of the person's face when worn by the person.

Clause 52 includes the computer-readable storage device of any of Clauses 49-51, wherein the output device includes an audio device.

Clause 53 includes the computer-readable storage device of any of Clauses 49-52, wherein the output device includes a display device configured to display an augmented reality (AR) heads-up display (HUD).

Clause 54 includes the computer-readable storage device of Clause 53, wherein the display device is external to a personal protective equipment (PPE) configured to cover at least a portion of the person's face when worn by the person.

Clause 55 includes the computer-readable storage device of Clause 53, wherein the display device is part of a personal protective equipment (PPE) configured to cover at least a portion of the person's face when worn by the person, disposed within the PPE, or both.

Clause 56 includes the computer-readable storage device of Clause 54 or Clause 55, wherein the PPE includes a helmet.

Clause 57 includes the computer-readable storage device of any of Clauses 54-56, wherein the PPE includes a mask or mask cover.

Clause 58 includes the computer-readable storage device of any of Clauses 54-57, wherein the PPE includes a face shield.

Clause 59 includes the computer-readable storage device of any of Clauses 54-58, wherein the PPE is compliant with an American National Standards Institute (ANSI) Z81 standard.

Clause 60 includes the computer-readable storage device of any of Clauses 46-59, wherein the disinfection device includes a lamp configured to output ultraviolet (UV) light.

Clause 61 includes the computer-readable storage device of Clause 60, wherein the UV light includes UV-C light, does not include UV-A light, and does not include UV-B light.

Clause 62 includes the computer-readable storage device of any of Clauses 46-61, wherein the disinfection device includes at least one of a chemical emitter, an aerosol emitter, an ultrasonic speaker, a microwave energy emitter, or a robotic device.

Clause 63 includes the computer-readable storage device of Clause 62, wherein the instructions, when executed by the one or more processors, further cause the one or more processors to determine whether to activate the disinfection device based at least in part on an output of a sensor.

Clause 64 includes the computer-readable storage device of Clause 63, wherein the sensor includes at least one of a thermal sensor, an optical sensor, an infrared sensor, a biosensor, a lab-on-chip sensor, or an airborne particle analysis sensor.

Clause 65 includes the computer-readable storage device of Clause 63 or Clause 64, wherein the instructions, when executed by the one or more processors, further cause the one or more processors to determine, based at least in part on an output of the sensor, a likelihood that a particular object or environment of the person is infected by a pathogen.

Clause 66 includes the computer-readable storage device of Clause 65, wherein the instructions, when executed by the one or more processors, further cause the one or more processors to generate activation data based at least in part on the likelihood.

Clause 67 includes the computer-readable storage device of Clause 66, wherein the instructions, when executed by the one or more processors, further cause the one or more processors to communicate information to the person via an output device, the information based at least in part on the likelihood.

According to Clause 68, a device includes means for receiving input data from an input device, the input data representative of an input from a person at the input device. The device also includes means for determining whether to activate a disinfection device configured to be worn or carried by the person based at least on the input data. The device also includes means for generating activation data based at least on the determination. The device also includes means for communicating activation data to the disinfection device, the activation data configured to selectively activate the disinfection device.

Clause 69 includes the device of Clause 68, wherein the input device includes at least one of a microphone or microphone array configured to receive speech input from the person or a tactile input device configured to receive tactile input from the person.

Clause 70 includes the device of Clause 68 or Clause 69, wherein the input device includes a network interface configured to receive input via a network.

Clause 71 includes the device of any of Clauses 68-70, wherein the device also includes means for communicating information to the person via an output device.

Clause 72 includes the device of Clause 71, wherein the information includes at least one of: a first instruction to place an object or a body part within a field of operation of the disinfection device; a second instruction to remove the object or the body part from within the field of operation of the disinfection device; or a third instruction to move the object or the body part while the object or the body part is in the field of operation of the disinfection device.

Clause 73 includes the device of Clause 71 or Clause 72, wherein the information includes information regarding a status of at least one of a power supply, the disinfection device, or a personal protective equipment (PPE) configured to cover at least a portion of the person's face when worn by the person.

Clause 74 includes the device of any of Clauses 71-73, wherein the output device includes an audio device.

Clause 75 includes the device of any of Clauses 71-74, wherein the output device includes a display device configured to display an augmented reality (AR) heads-up display (HUD).

Clause 76 includes the device of Clause 75, wherein the display device is external to a personal protective equipment (PPE) configured to cover at least a portion of the person's face when worn by the person.

Clause 77 includes the device of Clause 75, wherein the display device is part of a personal protective equipment (PPE) configured to cover at least a portion of the person's face when worn by the person, disposed within the PPE, or both.

Clause 78 includes the device of Clause 76 or Clause 77, wherein the PPE includes a helmet.

Clause 79 includes the device of any of Clauses 76-78, wherein the PPE includes a mask or mask cover.

Clause 80 includes the device of any of Clauses 76-79, wherein the PPE includes a face shield.

Clause 81 includes the device of any of Clauses 76-80, wherein the PPE is compliant with an American National Standards Institute (ANSI) Z81 standard.

Clause 82 includes the device of any of Clauses 68-81, wherein the disinfection device includes a lamp configured to output ultraviolet (UV) light.

Clause 83 includes the device of Clause 82, wherein the UV light includes UV-C light, does not include UV-A light, and does not include UV-B light.

Clause 84 includes the device of any of Clauses 68-83, wherein the disinfection device includes at least one of a chemical emitter, an aerosol emitter, an ultrasonic speaker, a microwave energy emitter, or a robotic device.

Clause 85 includes the device of Clause 84, wherein the means for determining whether to activate the disinfection device further includes means for determining whether to activate the disinfection device based at least in part on an output of a sensor.

Clause 86 includes the device of Clause 85, wherein the sensor includes at least one of a thermal sensor, an optical sensor, an infrared sensor, a biosensor, a lab-on-chip sensor, or an airborne particle analysis sensor.

Clause 87 includes the device of Clause 86, wherein the device also includes means for determining, based at least in part on an output of the sensor, a likelihood that a particular object or environment of the person is infected by a pathogen.

Clause 88 includes the device of Clause 87, wherein the means for generating activation data further includes means for generating activation data based at least in part on the likelihood.

Clause 89 includes the device of Clause 88, wherein the device also includes means for communicating information to the person via an output device, the information based at least in part on the likelihood.

Although the disclosure may include one or more methods, it is contemplated that it may be embodied as computer program instructions on a tangible computer-readable medium, such as a magnetic or optical memory or a magnetic or optical disk/disc. All structural, chemical, and functional equivalents to the elements of the above-described exemplary embodiments that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Changes and modifications may be made to the disclosed embodiments without departing from the scope of the present disclosure. These and other changes or modifications are intended to be included within the scope of the present disclosure, as expressed in the following claims.

What is claimed is:

1. A personal protection and pathogen disinfection system, comprising:
personal protective equipment (PPE) configured to cover at least a portion of a person's face when worn by the person;
a disinfection device configured to be worn or carried by the person;
an input device configured to receive input from the person; and
at least one processor configured to selectively activate the disinfection device responsive to the input.

2. The system of claim 1, wherein the PPE comprises a helmet, a mask, a mask cover, a face shield, or some combination thereof.

3. The system of claim 1, wherein the PPE is compliant with an American National Standards Institute (ANSI) Z81 standard.

4. The system of claim 1, wherein the input device comprises at least one of a microphone or microphone array configured to receive speech input from the person, a tactile input device configured to receive tactile input from the person, a network interface configured to receive input via a network, or a combination thereof.

5. The system of claim 1, further comprising an output device configured to communicate information to the person.

6. The system of claim 5, wherein the output device comprises an audio device.

7. The system of claim 5, wherein the output device comprises a display device configured to display an augmented reality (AR) heads-up display (HUD).

8. The system of claim 7, wherein the display device is external to the PPE.

9. The system of claim 8, wherein the display device is part of the PPE, disposed within the PPE, or both.

10. The system of claim 1, wherein the disinfection device comprises a lamp configured to output ultraviolet (UV) light, a chemical emitter, an aerosol emitter, an ultrasonic speaker, a microwave energy emitter, a robotic device, or a combination thereof.

11. The system of claim 10, wherein the UV light includes UV-C light, does not include UV-A light, and does not include UV-B light.

12. The system of claim 1, further comprising an output device configured to output at least one of:
a first instruction to place an object or a body part within a field of operation of the disinfection device;
a second instruction to remove the object or the body part from within the field of operation of the disinfection device; or
a third instruction to move the object or the body part while the object or the body part is in the field of operation of the disinfection device.

13. The system of claim 1, further comprising an output device configured to output information regarding a status of at least one of a power supply, the disinfection device, or the PPE.

14. The system of claim 1, further comprising a sensor, wherein the processor is further configured to selectively activate the disinfection device based at least in part on an output of the sensor.

15. The system of claim 14, wherein the sensor includes at least one of a thermal sensor, an optical sensor, an infrared sensor, a biosensor, a lab-on-chip sensor, or an airborne particle analysis sensor.

16. The system of claim 15, wherein the processor is configured to determine, based at least in part on an output of the sensor, a likelihood that a particular object or environment of the person is infected by a pathogen.

17. The system of claim 16, wherein the processor is further configured to selectively activate the disinfection device based at least in part on the likelihood.

18. The system of claim 17, further comprising an output device configured to output information based on the likelihood.

19. A method comprising:
receiving input data from an input device, the input data representative of an input from a person at the input device;
determining whether to activate a disinfection device configured to be worn or carried by the person based at least on the input data;
generating activation data based at least on the determination; and
communicating activation data to the disinfection device, the activation data configured to selectively activate the disinfection device.

20. A computer-readable storage device storing instructions that, when executed by one or more processors, cause the one or more processors to:
receive input data from an input device, the input data representative of an input from a person at the input device;
determine whether to activate a disinfection device configured to be worn or carried by the person based at least on the input data;
generate activation data based at least on the determination; and
communicate activation data to the disinfection device, the activation data configured to selectively activate the disinfection device.
